(19) United States
(12) Patent Application Publication
(10) Pub. No.: US 2009/0215676 A1
(43) Pub. Date: Aug. 27, 2009

(54) Method of treatment

(76) Inventor: Aart Jan VAN DER LELY, Rotterdam (NL)

Correspondence Address: Alan F. Feeny, Esq., Biomeasure, Incorporated, 27 Maple Street, Milford, MA, 01757-3650, US

(21) Appl. No.: 11/918,460

(22) PCT Filed: Apr. 13, 2006
(86) PCT No.: PCT/EP2006/003438

§ 371 Date: Feb. 13, 2009

Related U.S. Application Data
(60) Provisional application No. 60/670,740, filed on Apr. 13, 2005.

(51) Int. Cl. A61K 38/31; A61K 38/12

(52) U.S. Cl. 514/9; 514/12

(57) ABSTRACT

Disclosed is a method for attenuating the effects of growth hormone (GH) in a subject. The method comprises the administration of both a GH antagonist and a somatostatin agonist to said subject, simultaneously or separately, continuously or periodically. In one preferred embodiment of the method an extended release somatostatin agonist composition is administered monthly, and a conventional non-extended release GH antagonist composition is administered weekly.

### RELATED APPLICATIONS

This application claims priority under 35 U.S.C. section 119 of Application Ser. No. 60/670,740, filed Apr. 13, 2005.

### DESCRIPTION OF THE INVENTION

1. Field of the Invention

The present invention relates to a method and compositions useful for attenuating the effects of elevated levels of growth hormone (GH) in the blood. More particularly, the present invention relates to the reduction of GH levels and/or insulin-like growth factor-1 (IGF-1) levels in the blood by the administration of a combination of a GH antagonist with a somatostatin or somatostatin agonist.

1. Background of the Invention

Conditions related to GH excess are well known to medical practitioners. One of the best known examples of such conditions, acromegaly, is characterized by excessive levels of GH in the blood, often resulting from an adenoma of the anterior pituitary. Acromegaly is associated with significant risk of morbidity (soft-tissue swelling, arthralgia, headache, perspiration, fatigue, CV disorders), insulin resistance and diabetes, vision problems resulting from optic nerve compression by the adenoma, and premature mortality. Most of the biological impacts and symptoms related to GH excess are mediated through IGF-1, which is secreted by the liver as well as many other target organs as a result of GH receptor activation.

Traditional treatment options for acromegaly include surgical removal of the offending tumor with or without follow-on, and normally chronic, medical treatment with GH suppressive drugs. Common among such drugs are somatostatin analogs such as LANREOTIDE (Ipsen, Paris, France) and OCTREOTIDE (Novartis, Basle, Switzerland) and dopamine agonists such as bromocriptine, cabergoline, and pergolide. However the dopamine agonists, while generally effective at providing symptomatic relief, rarely normalize GH levels.

LANREOTIDE and OCTREOTIDE are able to reduce GH and IGF-1 levels in approximately 90% of acromegalic patients. Of these, approximately one-half to two-thirds are able to reduce their GH and IGF-1 levels to normal levels (“full responders”), while the rest are able to reduce their GH and IGF-1 levels, albeit not to normal levels (“partial responders”). A number of dosage forms for somatostatin analogs are already available for use or are otherwise well known to persons skilled in the art of pharmacy.

For those approximately 10% of acromegalic patients who experience no significant lowering of GH levels in response to somatostatin agonists treatment (“non-responders”), PEGVISOMANT (SOMAVERT, Pfizer, Inc., New York, USA), a representative of a new class of drugs (GH antagonists), has recently been made commercially available. PEGVISOMANT comprises a recombinantly produced, 191 amino acid analog of the GH protein to which polyethyleneglycol moieties have been attached (i.e., the protein has been subjected to “pegylation”). Various methods of producing recombinant proteins, and in particular, growth hormone antagonists, are well known to persons skilled in the art of pharmacy. (By way of example and not limitation, see U.S. Pat. Nos. 5,350,836; 5,681,809; 5,849,535; 5,958,879; 6,057,292; and 6,583,115; United States Patent Publication No.'s 20060026719; 20050214762; 20050123558; 20050059577; and 20040071655. See also Kopchick et al., Endocrine Reviews, (2002), 23(5) pp. 623-646.) Rather than targeting GH secretion, GH antagonists like PEGVISOMANT are believed to competitively bind to, but not activate, the GH receptor, thereby substantially attenuating most of the effects of high levels of circulating endogenous GH (i.e., normalize IGF-I levels) in most (e.g., 75%-95%) acromegalics. Pegylation of the GH antagonist protein is intended to improve in vivo half-life and reduce immunogenicity. (Kopchick et al., Endocrine Rev, (2002), 23(5) pp. 623-646.)

### SUMMARY OF THE INVENTION

Experience with the currently available GH antagonist demonstrates that, while this class of therapeutic agent may be generally effective at alleviating most negative effects of high circulating endogenous GH levels, relatively high in vivo concentrations of a GH antagonist are required in order to compete effectively for the GH receptor, hence a high dose must be administered (10 to 40 mg per day or higher in the case of Pegvisomant). Further, while pegylation of the GH antagonist protein offers improved in vivo half-life relative to the non-pegylated form, currently practice with PEGVISOMANT demonstrates that this type of therapeutic agent often needs to be administered on a daily basis in order to assure efficacy.

Although several groups have attempted to extend the dosing schedule, until the present invention it has not been demonstrated that a substantial reduction in the current daily dosing regimen of this highly expensive medication was possible for a significant proportion of acromegalic patients. (See, e.g., Jehle et al., J. Clin. Endo. Metab., 90(3):1588-1593 (2005); European Public Assessment Report, Scientific Discussion, (available at www.emea.eu.int/humandocs/Humans/EPAR/somavert/somavert.htm.) Thus prior to the present invention there remained a significant need for a method to reduce the dosage and/or the frequency at which a GH antagonist must be administered in order to effect a positive clinical outcome. Such reductions will result in concomtment reduction in the number of painful injections which patients currently must endure, improved quality of life, patient compliance, and

Thus in a first aspect, the invention relates to a method for attenuating the effects of GH in a human or non-human subject in whom such attenuation is desired, said method comprising administering to said subject both a GH antagonist and a somatostatin or a somatostatin agonist.

In a first embodiment of said first aspect said subject is a mammal. Preferably said mammal is a human being, more preferably a human being whose blood plasma level of GH is higher than desired, more preferably still a human being who is suffering from acromegaly or who is at risk of developing acromegaly or symptoms thereof.

More preferably with respect to said first embodiment of said first aspect, said subject is a human who suffers from acromegaly and said GH antagonist comprises PEGVISOMANT, wherein said PEGVISOMANT is administered with a frequency of between about once per day and about once every month, inclusive, preferably between about once every 3 days (i.e., about every 2, 3, or 4 days) and about once every 14 days, inclusive, more preferably about once per week, (i.e., about once every 5, 6, 7, 8, or 9 days), most preferably about once every 7 days.

Also more preferably with respect to said first embodiment of said first aspect, said subject is a human who suffers from acromegaly and said somatostatin agonist comprises OCTREOTIDE or LANREOTIDE, wherein said OCTREOTIDE or said LANREOTIDE is administered with a frequency of between about five times per day and about once every 6 months, inclusive, preferably between about three times per day and about once every 3 months, inclusive, more preferably between about once per day and about once per month, inclusive, more preferably about once per month. More preferably when said somatostatin agonist is OCTREOTIDE it is provided as OCTREOTIDE LAR, and when said somatostatin agonist is LANREOTIDE it is provided as LANREOTIDE AUTOGEL.

In a still more preferred embodiment of said first aspect, said human subject suffers from acromegaly, said GH antagonist comprises PEGVISOMANT, and said somatostatin agonist comprises OCTREOTIDE, wherein said PEGVISOMANT is administered about once per week and said OCTREOTIDE is administered about once per month.

In another still more preferred embodiment of said first aspect, said human subject suffers from acromegaly, said GH antagonist comprises PEGVISOMANT, and said somatostatin agonist comprises LANREOTIDE, wherein said PEGVISOMANT is administered about once per week and said LANREOTIDE is administered about once per month.

In a second aspect, the invention relates to a method for reducing the dose of GH antagonist needed to attenuate the effects of GH in a human or non-human subject in whom such attenuation is desired, said method comprising administering to said subject both a GH antagonist and a somatostatin or a somatostatin agonist.

In a first embodiment of said second aspect said subject is a mammal. Preferably said mammal is a human being, more preferably a human being whose blood plasma level of GH is higher than desired, more preferably still a human being who is suffering from acromegaly or who is at risk of developing acromegaly or symptoms thereof.

More preferably with respect to said first embodiment of said second aspect, said subject is a human who suffers from acromegaly and said GH antagonist comprises PEGVISOMANT, wherein said PEGVISOMANT is administered with a frequency of between about once per day and about once every month, inclusive, preferably between about once every 3 days (i.e., about every 2, 3, or 4 days) and about once every 14 days, inclusive, more preferably about once per week, (i.e., about once every 5, 6, 7, 8, or 9 days), most preferably about once every 7 days.

Also more preferably with respect to said first embodiment of said second aspect, said subject is a human who suffers from acromegaly and said somatostatin agonist comprises OCTREOTIDE or LANREOTIDE, wherein said OCTREOTIDE or said LANREOTIDE is administered with a frequency of between about five times per day and about once every 6 months, inclusive, preferably between about three times per day and about once every 3 months, inclusive, more preferably between about once per day and about once per month, inclusive, more preferably about once per month. More preferably when said somatostatin agonist is OCTREOTIDE it is provided as OCTREOTIDE LAR, and when said somatostatin agonist is LANREOTIDE it is provided as LANREOTIDE AUTOGEL.

In a still more preferred embodiment of said second aspect, said human subject suffers from acromegaly, said GH antagonist comprises PEGVISOMANT, and said somatostatin agonist comprises OCTREOTIDE, wherein said PEGVISOMANT is administered about once per week and said OCTREOTIDE is administered about once per month.

In another still more preferred embodiment of said second aspect, said human subject suffers from acromegaly, said GH antagonist comprises PEGVISOMANT, and said somatostatin agonist comprises LANREOTIDE, wherein said PEGVISOMANT is administered about once per week and said LANREOTIDE is administered about once per month.

In a third aspect, the invention relates to a method for reducing the frequency of administration of GH antagonist needed to attenuate the effects of GH in a human or non-human subject in whom such attenuation is desired, said method comprising administering to said subject both a GH antagonist and a somatostatin or a somatostatin agonist.

In a first embodiment of said third aspect said subject is a mammal. Preferably said mammal is a human being, more preferably a human being whose blood plasma level of GH is higher than desired, more preferably still a human being who is suffering from acromegaly or who is at risk of developing acromegaly or symptoms thereof.

More preferably with respect to said first embodiment of said third aspect, said subject is a human who suffers from acromegaly and said GH antagonist comprises PEGVISOMANT, wherein said PEGVISOMANT is administered with a frequency of between about once per day and about once every month, inclusive, preferably between about once every 3 days (i.e., about every 2, 3, or 4 days) and about once every 14 days, inclusive, more preferably about once per week, (i.e., about once every 5, 6, 7, 8, or 9 days), most preferably about once every 7 days.

Also more preferably with respect to said first embodiment of said third aspect, said subject is a human who suffers from acromegaly and said somatostatin agonist comprises OCTREOTIDE or LANREOTIDE, wherein said OCTREOTIDE or said LANREOTIDE is administered with a frequency of between about five times per day and about once every 6 months, inclusive, preferably between about three times per day and about once every 3 months, inclusive, more preferably between about once per day and about once per month, inclusive, more preferably about once per month. More preferably when said somatostatin agonist is OCTREOTIDE it is provided as OCTREOTIDE LAR, and when said somatostatin agonist is LANREOTIDE it is provided as LANREOTIDE AUTOGEL.

In a still more preferred embodiment of said third aspect, said human subject suffers from acromegaly, said GH antagonist comprises PEGVISOMANT, and said somatostatin agonist comprises OCTREOTIDE, wherein said PEGVISOMANT is administered about once per week and said OCTREOTIDE is administered about once per month.

In another still more preferred embodiment of said third aspect, said human subject suffers from acromegaly, said GH antagonist comprises PEGVISOMANT, and said somatostatin agonist comprises LANREOTIDE, wherein said PEGVISOMANT is administered about once per week and said LANREOTIDE is administered about once per month.

In a fourth aspect, the invention relates to a method for normalizing serum IGF-I concentration in a human or non-human subject in whom such normalization is desired, said method comprising administering to said subject both a GH antagonist and a somatostatin or a somatostatin agonist.

In a first embodiment of said fourth aspect said subject is a mammal. Preferably said mammal is a human being, more preferably a human being whose blood plasma level of GH is higher than desired, more preferably still a human being who is suffering from acromegaly or who is at risk of developing acromegaly or symptoms thereof.

More preferably with respect to said first embodiment of said fourth aspect, said subject is a human who suffers from acromegaly and said GH antagonist comprises PEGVISOMANT, wherein said PEGVISOMANT is administered with a frequency of between about once per day and about once every month, inclusive, preferably between about once every 3 days (i.e., about every 2, 3, or 4 days) and about once every 14 days, inclusive, more preferably about once per week, (i.e., about once every 5, 6, 7, 8, or 9 days), most preferably about once every 7 days.

Also more preferably with respect to said first embodiment of said fourth aspect, said subject is a human who suffers from acromegaly and said somatostatin agonist comprises OCTREOTIDE or LANREOTIDE, wherein said OCTREOTIDE or said LANREOTIDE is administered with a frequency of between about five times per day and about once every 6 months, inclusive, preferably between about three times per day and about once every 3 months, inclusive, more preferably between about once per day and about once per month, inclusive, more preferably about once per month. More preferably when said somatostatin agonist is OCTREOTIDE it is provided as OCTREOTIDE LAR, and when said somatostatin agonist is LANREOTIDE it is provided as LANREOTIDE AUTOGEL.

In a still more preferred embodiment of said fourth aspect, said human subject suffers from acromegaly, said GH antagonist comprises PEGVISOMANT, and said somatostatin agonist comprises OCTREOTIDE, wherein said PEGVISOMANT is administered about once per week and said OCTREOTIDE is administered about once per month.

In another still more preferred embodiment of said fourth aspect, said human subject suffers from acromegaly, said GH antagonist comprises PEGVISOMANT, and said somatostatin agonist comprises LANREOTIDE, wherein said PEGVISOMANT is administered about once per week and said LANREOTIDE is administered about once per month.

In a fifth aspect, the invention relates to a method for reducing the dose of GH antagonist needed to normalize the serum IGF-I concentration in a human or non-human subject in whom such normalization is desired, said method comprising administering to said subject both a GH antagonist and a somatostatin or a somatostatin agonist.

In a first embodiment of said fifth aspect said subject is a mammal. Preferably said mammal is a human being, more preferably a human being whose blood plasma level of GH is higher than desired, more preferably still a human being who is suffering from acromegaly or who is at risk of developing acromegaly or symptoms thereof.

More preferably with respect to said first embodiment of said fifth aspect, said subject is a human who suffers from acromegaly and said GH antagonist comprises PEGVISOMANT, wherein said PEGVISOMANT is administered with a frequency of between about once per day and about once every month, inclusive, preferably between about once every 3 days (i.e., about every 2, 3, or 4 days) and about once every 14 days, inclusive, more preferably about once per week, (i.e., about once every 5, 6, 7, 8, or 9 days), most preferably about once every 7 days.

Also more preferably with respect to said first embodiment of said fifth aspect, said subject is a human who suffers from acromegaly and said somatostatin agonist comprises OCTREOTIDE or LANREOTIDE, wherein said OCTREOTIDE or said LANREOTIDE is administered with a frequency of between about five times per day and about once every 6 months, inclusive, preferably between about three times per day and about once every 3 months, inclusive, more preferably between about once per day and about once per month, inclusive, more preferably about once per month. More preferably when said somatostatin agonist is OCTREOTIDE it is provided as OCTREOTIDE LAR, and when said somatostatin agonist is LANREOTIDE it is provided as LANREOTIDE AUTOGEL.

In a still more preferred embodiment of said fifth aspect, said human subject suffers from acromegaly, said GH antagonist comprises PEGVISOMANT, and said somatostatin agonist comprises OCTREOTIDE, wherein said PEGVISOMANT is administered about once per week and said OCTREOTIDE is administered about once per month.

In another still more preferred embodiment of said fifth aspect, said human subject suffers from acromegaly, said GH antagonist comprises PEGVISOMANT, and said somatostatin agonist comprises LANREOTIDE, wherein said PEGVISOMANT is administered about once per week and said LANREOTIDE is administered about once per month.

In a sixth aspect, the invention relates to a method for reducing the frequency of administration of GH antagonist needed to normalize the serum IGF-I concentration in a human or non-human subject in whom such normalization is desired, said method comprising administering to said subject both a GH antagonist and a somatostatin or a somatostatin agonist.

In a first embodiment of said sixth aspect said subject is a mammal. Preferably said mammal is a human being, more preferably a human being whose blood plasma level of GH is higher than desired, more preferably still a human being who is suffering from acromegaly or who is at risk of developing acromegaly or symptoms thereof.

More preferably with respect to said first embodiment of said sixth aspect, said subject is a human who suffers from acromegaly and said GH antagonist comprises PEGVISOMANT, wherein said PEGVISOMANT is administered with a frequency of between about once per day and about once every month, inclusive, preferably between about once every 3 days (i.e., about every 2, 3, or 4 days) and about once every 14 days, inclusive, more preferably about once per week, (i.e., about once every 5, 6, 7, 8, or 9 days), most preferably about once every 7 days.

Also more preferably with respect to said first embodiment of said sixth aspect, said subject is a human who suffers from acromegaly and said somatostatin agonist comprises OCTREOTIDE or LANREOTIDE, wherein said OCTREOTIDE or said LANREOTIDE is administered with a frequency of between about five times per day and about once every 6 months, inclusive, preferably between about three times per day and about once every 3 months, inclusive, more preferably between about once per day and about once per month, inclusive, more preferably about once per month. More preferably when said somatostatin agonist is OCTREOTIDE it is provided as OCTREOTIDE LAR, and when said somatostatin agonist is LANREOTIDE it is provided as LANREOTIDE AUTOGEL.

In a still more preferred embodiment of said sixth aspect, said human subject suffers from acromegaly, said GH antagonist comprises PEGVISOMANT, and said somatostatin agonist comprises OCTREOTIDE, wherein said PEGVISOMANT is administered about once per week and said OCTREOTIDE is administered about once per month.

In another still more preferred embodiment of said sixth aspect, said human subject suffers from acromegaly, said GH antagonist comprises PEGVISOMANT, and said somatostatin agonist comprises LANREOTIDE, wherein said PEGVISOMANT is administered about once per week and said LANREOTIDE is administered about once per month.

### BRIEF DESCRIPTION OF THE DRAWING

FIG. 1: Normalization of serum IGF-I concentration in 19 acromegalic patients before and after adding once weekly pegvisomant to high-dose monthly somatostatin analogue therapy in a dose finding study with a treatment duration of 42 weeks and dose increments to a maximum of 80 mg of pegvisomant per week. The shaded area indicates the age-dependent normal ranges for IGF-I.

### DETAILED DESCRIPTION OF THE INVENTION

### EXAMPLE 1

We examined in a 42-week dose-finding study the efficacy of the combination of long-acting somatostatin analogues once monthly and pegvisomant once weekly in 26 patients with active acromegaly. Pegvisomant dose was increased until IGF-I levels normalized or until a weekly dose of 80 mg was reached. IGF-I levels normalized in 25 (95%) with a median weekly dose of 60 mg pegvisomant. There were no signs of pituitary tumor growth but mild elevations in liver enzymes were observed in 10 patients (38%). The combined therapy might increase compliance, while it can significantly reduce the costs of medical therapy.

Long-acting somatostatin analogues normalize serum IGF-I levels in two-third of patients (1). Pegvisomant normalizes IGF-I levels in >90% (2;3). We performed an investigator-initiated, 42 weeks, single-centre prospective open label dose-finding study in which 26 acromegalic patients were treated with both a long-acting somatostatin analog and weekly administration of pegvisomant. These patients could not be controlled with long-acting somatostatin analogue monotherapy during at least six months preceding the date of study entrance. The study was approved by the local medical ethical committee and all patients gave their consent. All patients were seen at 6 weeks intervals. Monthly 30 mg of octreotide LAR (also known as SANDOSTATIN LAR) or 120 mg of Lanreotide autogel (also known as SOMATULINE AUTOGEL) therapy was continued. Starting dose of pegvisomant was 25 mg per week. Pegvisomant dosage was adjusted until serum IGF-I concentrations were within the age-adjusted normal range. Efficacy was determined at week 42, which was 6 weeks after patients could have reached the maximal allowed dose of 80 mg when necessary. Efficacy parameters were assessed just prior to the next weekly pegvisomant administration. Serum IGF-I concentration were measured by an immunometric assay (Diagnostic Products Corporation; Los Angeles, USA). A Wilcoxon's signed rank test was used for assessing significance of changes from baseline. Statistical significance was accepted at p-values<0.05.

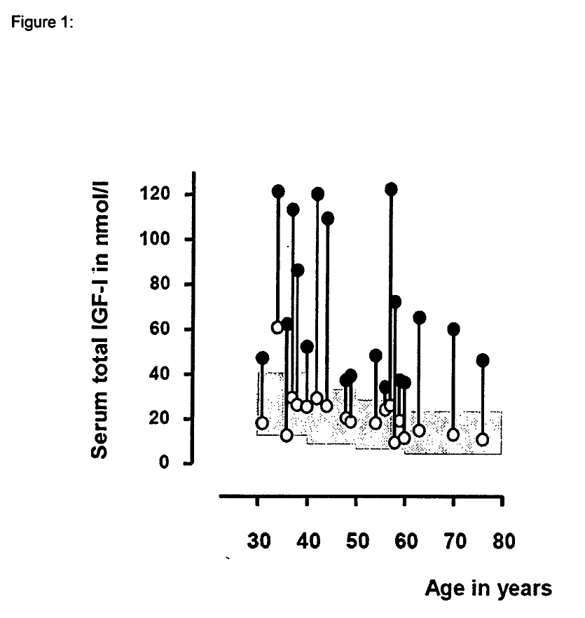

Table I shows the baseline characteristics. After 18 weeks treatment with pegvisomant (i.e. with at least 50 mg of pegvisomant per week), normalization of serum IGF-I concentrations could be achieved in 21 of the 26 subjects (81%). At week 42 (n=19 of 26), IGF-I levels were normalized in 95%. Mean serum IGF-I decreased from 67.7±29.9 nmol/l at baseline to a lowest value of 24.4±12.0 nmol/l with combined treatment (see FIG. 1 for individual changes). Median dose of weekly pegvisomant necessary to normalize serum IGF-I concentration was 60 mg. Interestingly, in a phase II study in the past (unpublished data) a once weekly pegvisomant dose of 80 mg was only effective in normalizing IGF-I in less than one-third of the patients. No signs of pituitary tumor growth were observed on MRI in those subjects who had completed a six-months treatment period (n=19). In 16% of the patients tumor regression could be demonstrated by an independent neuroradiologist (using the surface to volume summation method), including 18 patients who never had received radiotherapy. Although the data on follow-up with pegvisomant monotherapy to date do not indicate that pegvisomant increases mean tumor size, clinical experience shows that pegvisomant treatment at least does not prevent tumor growth in some patients (4). Mild pegvisomant-dose independent and non-progressive elevations in liver transaminases were observed in 10 patients (38%). There were no drop-outs.

In this proof of principle study we have demonstrated that in patients in whom serum IGF-I levels can not be controlled by monthly long-acting somatostatin analogue monotherapy, normalization of serum IGF-I can be obtained by adding weekly pegvisomant. The observed 95% efficacy is equal to that of daily pegvisomant monotherapy. Weekly instead of daily injections might improve patients' compliance. Also, combined therapy is potentially considerably cheaper than pegvisomant monotherapy, at least in some patients. The average patient treated with pegvisomant monotherapy needs approximately 20 mg daily. We have calculated that combination therapy will be equally expensive as daily 20 mg pegvisomant monotherapy when a weekly pegvisomant dose of 65 mg in the combination treatment regimen is used. Median weekly pegvisomant dosage for normalizing serum IGF-I concentration was 60 mg in our study. In the present study, however, three subjects also participated in one of the former pegvisomant registration studies. Two of them needed 40 mg and one needed 35 mg of pegvisomant daily. In the present study, their IGF-I has normalized with pegvisomant 60 mg and 80 mg weekly, respectively. For patients who need 40 mg of daily pegvisomant monotherapy, the combination therapy could save ≈C=58 thousand (≈UK£ 40.3 thousand; =US$ 75.4 thousand) on an annual basis. Recently, a study was published on alternate day administration of pegvisomant monotherapy (5). This regimen failed to maintain IGF-I within the age-adjusted normal range in 7 of 10 patients. Apparently, most patients treated with pegvisomant monotherapy require daily administration (5). Pegvisomant monotherapy improves insulin sensitivity as compared to somatostatin analogues (6). Although we have not studied this particular issue, one might expect that pegvisomant monotherapy, compared to the combination therapy, has beneficial effects on insulin sensitivity, as somatostatin analogues decrease insulin sensitivity (6).

Less pegvisomant is needed when there is less GH to compete with, e.g. during co-treatment with a somatostatin analogue. Also,. lower insulin levels in the portal vein, with somatostatin analog therapy, will decrease the number of available GH receptors at the cell surface of the hepatocytes (7). Somatostatin analogues might also increase pegvisomant levels by unknown mechanisms (4).

We conclude that combined treatment with monthly high-dose long-acting somatostatin analogue therapy and weekly subcutaneous pegvisomant administrations is as effective as daily pegvisomant monotherapy.

### REFERENCE LIST

1. Sheppard M C. Primary medical therapy for acromegaly. Clin. Endocrinol. (Oxf) 2003; 58(4):387-99.

2. Trainer P J, Drake W M, Katznelson L, Freda P U, Herman-Bonert V, van der Lely A J et al. Treatment of acromegaly with the growth hormone-receptor antagonist pegvisomant. N. Engl. J. Med. 2000; 342(16):1171-7.

3. van der Lely A J, Hutson R K, Trainer P J, Besser G M, Barkan A L, Katznelson L et al. Long-term treatment of acromegaly with pegvisomant, a growth hormone receptor antagonist. Lancet 2001; 358(9295):1754-9.

4. van der Lely A J, Muller A, Janssen J A, Davis R J, Zib K A, Scarlett J A et al. Control of tumor size and disease activity during cotreatment with octreotide and the growth hormone receptor antagonist pegvisomant in an acromegalic patient. J. Clin. Endocrinol. Metab 2001; 86(2):478-81.

5. Jehle S, Reyes C M, Sundeen R E, Freda P U. Alternate-day administration of pegvisomant maintains normal serum insulin-like growth factor-I levels in patients with acromegaly. J. Clin. Endocrinol. Metab 2005; 90(3):1588-93.

6. Drake W M, Rowles S V, Roberts M E, Fode F K, Besser G M, Monson J P et al. Insulin sensitivity and glucose tolerance improve in patients with acromegaly converted from depot octreotide to pegvisomant. Eur. J. Endocrinol. 2003; 149(6):521-7.

7. Leung K C, Doyle N. Ballesteros M, Waters M J, Ho K K. Insulin regulation of human hepatic growth hormone receptors: divergent effects on biosynthesis and surface translocation. J. Clin. Endocrinol. Metab 2000; 85(12):4712-20.

TABLE 1
Baseline characteristics (n = 26)

| | |
|---|---|
| Mean age | 51 yrs (31-79 yrs) (SD 12.6 yrs) |

Age distribution

| <40 yrs | 5 | (19%) |
|---|---|---|
| 40-49 yrs | 9 | (35%) |
| 50-59 yrs | 7 | (27%) |
| ≧60 yrs | 5 | (19%) |
| Gender | 15 male (58%) 11 female | |
| Baseline IGF-I | Mean: 66.7 nmol/L, SD 29.9 nmol/L | |
| | Median: 61.1 nmol/L, range 34-122 | |
| 1-2 ULN | 12 | (46%) |
| 2-3 ULN | 9 | (35%) |
| >3 ULN | 5 | (19%) |

| | |
|---|---|
| Baseline GH | Mean: 10.5 μg/L, SD 15.3 μg/L |
| | Median: 5.2 μg/L, range 0.4-69.8 |

Pre-treatment

| | | |
|---|---|---|
| Both TNH and RTx | 8 | (31%) |
| Only TNH | 4 | (15%) |
| Neither TNH nor RTx | 14 | (54%) |

Pituitary insufficiency

| | | |
|---|---|---|
| Panhypopituitarism | 6 | (23%) |
| No hypopituitarism | 9 | (35%) |
| 1-2 axes insufficient | 11 | (42%) |

Long-acting SRIF analogs

| | | |
|---|---|---|
| Lanreotide autosolution | 21 | (81%) |
| Octreotide LAR | 5 | (19%) |

Pituitary adenoma size

| | | |
|---|---|---|
| Macro adenoma | 12 | (46%) |
| Micro adenoma | 14 | (54%) |

Baseline characteristics of 26 patients with biochemically active acromegaly, despite long-term treatment with high-dose somatostatin analogues. (ULN = Upper level of normality; TNH = transnasal hypophysectomy; RTx = radiotherapy)

What is claimed is:

1. A method for attenuating the effects of growth hormone in a human or non-human subject in whom such attenuation is desired, said method comprising administering to said subject both a growth hormone antagonist and a somatostatin or a somatostatin agonist.

2. A method for reducing the dose of growth hormone antagonist needed to attenuate the effects of growth hormone in a human or non-human subject in whom such attenuation is desired, said method comprising administering to said subject both a growth hormone antagonist and a somatostatin or a somatostatin agonist.

3. A method for reducing the frequency of administration of growth hormone antagonist needed to attenuate the effects of growth hormone in a human or non-human subject in whom such attenuation is desired, said method comprising administering to said subject both a growth hormone antagonist and a somatostatin or a somatostatin agonist.

4. A method for normalizing serum IGF-I concentration in a human or non-human subject in whom such normalization is desired, said method comprising administering to said subject both a growth hormone antagonist and a somatostatin or a somatostatin agonist.

5. A method for reducing the dose of growth hormone antagonist needed to normalize the serum IGF-I concentration in a human or non-human subject in whom such normalization is desired, said method comprising administering to said subject both a growth hormone antagonist and a somatostatin or a somatostatin agonist.

6. A method for reducing the frequency of administration of growth hormone antagonist needed to normalize the serum IGF-I concentration in a human or non-human subject in whom such normalization is desired, said method comprising administering to said subject both a growth hormone antagonist and a somatostatin or a somatostatin agonist.

7. The method according to any one of claims 1-6, wherein said subject is human.

8. The method according to claim 7, wherein said human subject has or is at risk of developing symptoms of acromegaly.

9. The method according to claim 8, wherein said growth hormone antagonist comprises PEGVISOMANT.

10. The method according to claim 9, wherein said somatostatin agonist comprises OCTREOTIDE or LANREOTIDE.

11. The method according to claim 10, wherein said OCTREOTIDE or said LANREOTIDE is administered with a frequency of between about once every day and about once every month.

12. The method according to claim 11, wherein said OCTREOTIDE or said LANREOTIDE is administered about once every month.

13. The method according to claim 12, wherein said PEGVISOMANT is administered with a frequency of between about once every other day and about once every seven days.

14. The method according to claim 13, wherein said PEGVISOMANT is administered with a frequency of about once every seven days.

15. The method according to claim 14, wherein said OCTREOTIDE is administered as OCTREOTIDE-LAR.

16. The method according to claim 14, wherein said LANREOTIDE is administered as LANREOTIDE AUTOGEL.
